(19) United States
(12) Patent Application Publication
(10) Pub. No.: US 2026/0106016 A1
(43) Pub. Date: Apr. 16, 2026

(54) SYSTEMS AND METHODS FOR TREATING EATING DISORDERS USING VIRTUAL OR AUGMENTED REALITY

(71) Applicants: Christina RALPH NEARMAN, Louisville, KY (US); Cheri A. LEVINSON, Louisville, KY (US); UNIVERSITY OF LOUISVILLE RESEARCH FOUNDATION, INC., Louisville, KY (US)

(72) Inventors: Christina RALPH NEARMAN, Louisville, KY (US); Cheri A. LEVINSON, Louisville, KY (US)

(21) Appl. No.: 19/115,561

(22) PCT Filed: Sep. 25, 2023
(86) PCT No.: PCT/US2023/033619

§ 371 Date: Mar. 26, 2025

Related U.S. Application Data
(60) Provisional application No. 63/377,220, filed on Sep. 27, 2022.

(51) Int. Cl. G16H 20/70; G06T 19/20; G16H 50/20; G16H 50/30; G16H 50/50

(52) U.S. Cl. CPC G16H 20/70; G06T 19/20; G16H 50/20; G16H 50/30; G16H 50/50; G06T 2210/41; G06T 2215/16; G06T 2219/2021

(57) ABSTRACT

Systems (100) and methods (400; 500) to treat fears related to an eating disorder are provided. A humanoid computer object (110) that looks similar to a patient is created. During a treatment session, the object is presented to or created by the patient in a virtual reality environment that is displayed to the patient using goggles (420). The anxiety level of the patient is monitored (410; 430), and once it is within a reference level, the object transitions to look like the patient but with a change such as a noticeable weight gain (450). The patient can view and interact with the object from all angles. Depending on the anxiety level of the patient, additional changes to the object may be displayed, with each object having a greater associated change. Example changes may include changes related to eating disorder-specific fears such as weight gain, uncomfortable bodily sensations, and judgement.

### CROSS-REFERENCE TO RELATED APPLICATIONS

This application claims priority to U.S. Provisional Ser. No. 63/377,220 filed on Sep. 27, 2022. The disclosure of which is hereby incorporated by reference in its entirety.

### GOVERNMENT SUPPORT CLAUSE

This invention was made with government support under Grant No. R34 MH128213-01 awarded by the National Institutes of Health. The government has certain rights in the invention.

### BACKGROUND

Eating disorders are among the most serious and deadly of psychiatric illnesses. It is estimated that one person dies every fifty-two minutes directly from an eating disorder. Despite the high mortality, morbidity, societal costs, and relapse, only ˜50% of individuals respond to the current gold standard treatments, and ˜50% of those individuals then relapse within about two months of treatment.

Anxiety and fear are highly prevalent for individuals with eating disorders. Up to 85% of individuals with an eating disorder also meet one or more criteria for an anxiety disorder, and eating disorder specific fears (e.g., fear of weight gain, discomfort with bodily sensations, etc.) and anxiety are hallmark symptoms of eating disorders. Despite the centrality of fear in eating disorders, there are no products designed to address eating disorder related fears.

### SUMMARY

To solve these and other problems, a virtual reality or augmented reality-based system for the treatment of anxieties and fears associated with eating disorders is provided. Example fears may include the fear of weight gain, the fear of being embarrassed because of their weight, the fear of discomfort with uncomfortable body sensations (e.g., bloating, jiggly body areas, and thighs rubbing together), and the fear of eating in front of others. A “humanoid computer object” that resembles a patient is created. The patient is asked to wear virtual reality goggles during a treatment session. For treatment of the fear of weight gain and fear of discomfort of bodily sensations, during the treatment session the humanoid computer object is first rendered to the patient in a virtual environment with the patient able to choose gender, skin and hair color/hue/tone, overall body size/shape, as well as selecting individual body areas to change the size and shape of (e.g., upper arms, abdomen, love handles/hips, chest, thighs, buttocks, etc.) to personalize the humanoid to best match their own body and appearance. The patient can view the rendering of the humanoid computer object in the virtual environment from all angles, including looking down at ‘their’ personalized humanoid.’

Later, an anxiety level of the patient is assessed using one or more questions or by measurements taken by one or more physiological sensors. If the anxiety is below a threshold, indicating that the patient is not experiencing higher than normal anxiety, the humanoid computer object is re-rendered in the virtual environment with a weight, size, jiggly areas, and bodily sensations, audio instruction, and visual changes that are greater than the current weight and size, or intensified jiggle of growing body areas and bodily sensations of the patient. For example, the patient may be encouraged to drink in a gamified fashion both actually and with the humanoid, and visually see, and be instructed to attend to the uncomfortable sensations of bloating, growing larger, and feeling full.

After displaying the humanoid computer object with the greater weight and size (and/or intensified jiggly body with movement, and growing and touching body areas), the patient may be encouraged to interact with or view the humanoid computer object in the virtual environment. In addition, a treatment provider may engage with the patient through questions and the anxiety of the patient may be reassessed. Once the measured anxiety of the patient falls below the threshold anxiety, the size and weight of the humanoid computer object may be further increased, and the patient may be encouraged to drink or eat more and may be instructed to notice their bodily changes, sensations, and engage in their eating disorder specific fears (e.g., discomfort with feeling full, fear of weight gain, etc.). The process may repeat during the treatment sessions, or across multiple treatment sessions, until the measured anxiety levels of the patient indicate that the patient's eating disorder specific fears (e.g., fear of gaining weight, fears of uncomfortable bodily sensations, fears of judgement, fears of eating in front of others, etc.) are reduced.

In some aspects, the techniques described herein relate to an augmented-reality or virtual-reality system including a processor; a memory operatively coupled to the processor, the memory having instructions stored thereon for treating eating disorders, wherein execution of the instructions by the processor, cause the processor to: continuously render a first humanoid computer object in an augmented- or virtual-reality environment selected from a plurality of humanoid bodies defined at least by a gender type, body type, skin pigment, and associated weight, including a first selected body having an associated first weight value and a likeliness in appearance to the user; stop rendering of the first humanoid computer object based on a stop condition associated with an anxiety level value or score received from the user; and continuously render a second humanoid computer object in the augmented- or virtual-reality environment selected from the plurality of humanoid bodies, including a second selected body having an associated second weight value and a likeliness in appearance to the user, wherein the second weight value is greater than the first weight value.

In some aspects, the techniques described herein relate to an augmented-reality or virtual-reality system, wherein the user is sequentially presented a series of humanoid computer objects in the augmented- or virtual-reality environment, the series of humanoid computer objects having (i) a predefined number of selected bodies having a low weight value and a likeliness in appearance to the user, (ii) a current predefined number of selected bodies having a current weight value and a likeliness in appearance to the user, and (iii) a predefined number of selected bodies having a high weight value and a likeliness in appearance to the user.

In some aspects, the techniques described herein relate to an augmented-reality or virtual-reality system, wherein the low weight value, the current weight value, and the high weight value are selectable based on a percentage of the current weight of the patient.

In some aspects, the techniques described herein relate to an augmented-reality or virtual-reality system, wherein the low weight value, the current weight value, and the high weight value are selectable based on a pre-defined offset of the current weight of the patient.

In some aspects, the techniques described herein relate to an augmented-reality or virtual-reality system, wherein the first humanoid computer object is presented from a view of a camera defined in the augmented- or virtual-reality environment, wherein the camera is co-located to the first humanoid computer object (e.g., wherein the camera is movable by the augmented reality or virtual-reality system input to show different perspective and point of view of the first humanoid computer object to the user).

In some aspects, the techniques described herein relate to an augmented-reality or virtual-reality system, wherein the camera has a pre-defined height level that limits presentation of the first humanoid computer object to exclude a facial region of the first humanoid computer object.

In some aspects, the techniques described herein relate to an augmented-reality or virtual-reality system, wherein the first humanoid computer object has a blocking mosaic over a facial region of the first humanoid computer object.

In some aspects, the techniques described herein relate to an augmented-reality or virtual-reality system claim 1, wherein the system includes an AR-VR goggle.

In some aspects, the techniques described herein relate to a method of conducting an eating disorder therapy, the method including: establishing a baseline anxiety level value or score for a patient at a commencement of an eating disorder therapy; presenting, via a processor executing an augmented- or virtual-reality environment, to a patient, a first humanoid computer object in the augmented- or virtual-reality environment selected from a plurality of humanoid bodies defined at least by a gender type, body type, skin pigment, and associated weight, wherein the first humanoid computer object includes a first selected body having a likeliness in appearance to the patient; receiving, via the processor, a selection of a treatment module of a plurality of treatment modules, wherein each treatment module treats a different fear associated with an eating disorder; receiving an anxiety level value or score from the patient and upon the anxiety level value or score being within a pre-defined threshold of the baseline anxiety level value or score, stop presentation of the first humanoid computer object; and presenting, via the processor, a second humanoid computer object in the augmented- or virtual-reality environment selected, wherein the second humanoid computer object includes at least one change based on the fear associated with the selected treatment module.

In some aspects, the techniques described herein relate to a method, wherein the fear associated with the selected treatment module is a fear of weight gain, and at the at least one change to the second humanoid computer object includes an increased weight of the second humanoid computer object in contrast with the first humanoid computer object.

In some aspects, the techniques described herein relate to a method, wherein the fear associated with the selected treatment module is a fear of bodily sensations, and at the at least one change to the second humanoid computer object includes one or more of increased bloating or of increased jiggling in contrast with the first humanoid computer object.

In some aspects, the techniques described herein relate to a method, wherein the fear associated with the selected treatment module is a fear of weight gain, and at the at least one change to the second humanoid computer object includes a weight gain to selected body part of area of the second humanoid computer object in contrast with the first humanoid computer object.

In some aspects, the techniques described herein relate to a method, further including: detecting, via the processor, that that the user has consumed a product; and in response to the determination, presenting, via the processor, a third humanoid computer object in the augmented- or virtual-reality environment selected, wherein the third humanoid computer object includes at least one change based on the fear associated with the selected treatment module.

In some aspects, the techniques described herein relate to a non-transitory computer readable medium having instructions stored thereon for a virtual-reality software for eating disorder therapy, wherein execution of the instructions by the processor causes the processor to: continuously render a first humanoid computer object in an augmented- or virtual-reality environment selected from a plurality of humanoid bodies defined at least by a gender type, body type, skin pigment, and associated weight, including a first selected body having an associated first weight value and a likeliness in appearance to the user; stop rendering of the first humanoid computer object based on a stop condition associated with an anxiety level value or score received from the user; and continuously render a second humanoid computer object in the augmented- or virtual-reality environment selected from the plurality of humanoid bodies, including a second selected body having an associated second weight value and a likeliness in appearance to the user, wherein the second weight value is greater than the first weight value.

In some aspects, the techniques described herein relate to a computer readable medium, wherein the user is sequentially presented a series of humanoid computer objects in the augmented- or virtual-reality environment, the series of humanoid computer objects having (i) a predefined number of selected bodies having a low weight value and a likeliness in appearance to the user, (ii) a current predefined number of selected bodies having a current weight value and a likeliness in appearance to the user, and (iii) a predefined number of selected bodies having a high weight value and a likeliness in appearance to the user.

In some aspects, the techniques described herein relate to a computer readable medium, wherein the low weight value, the current weight value, and the high weight value are selectable based on a percentage of the current weight of the patient.

In some aspects, the techniques described herein relate to a computer readable medium, wherein the low weight value, the current weight value, and the high weight value are selectable based on a pre-defined offset of the current weight of the patient.

In some aspects, the techniques described herein relate to a computer readable medium, wherein the first humanoid computer object is presented from a view of a camera defined in the augmented- or virtual-reality environment, wherein the camera is co-located to the first humanoid computer object.

In some aspects, the techniques described herein relate to a computer readable medium, wherein the camera has a pre-defined height level that limits presentation of the first humanoid computer object to exclude a facial region of the first humanoid computer object.

In some aspects, the techniques described herein relate to a computer readable medium, wherein the first humanoid computer object has a blocking mosaic over a facial region of the first humanoid computer object.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying figures, which are incorporated herein and form part of the specification, illustrate an eating-disorder-related fear treatment system and method. Together with the description, the figures further serve to explain the principles of the eating disorder-related fear treatment system and method described herein and thereby enable a person skilled in the pertinent art to make and use the eating-disorder-related fear treatment system and method.

FIG. 1 is an example system for treating one or more fears related to an eating disorder;

FIG. 2 is an illustration of example humanoid computer objects;

FIGS. 3A and 3B are illustrations of example images of a virtual environment;

FIG. 4 is an illustration of a method for treating one or more fears associated with an eating disorder;

FIG. 5 is an illustration of a method for treating one or more fears associated with an eating disorder;

FIG. 6 shows an example computing environment in which example embodiments and aspects may be implemented.

### DETAILED DESCRIPTION

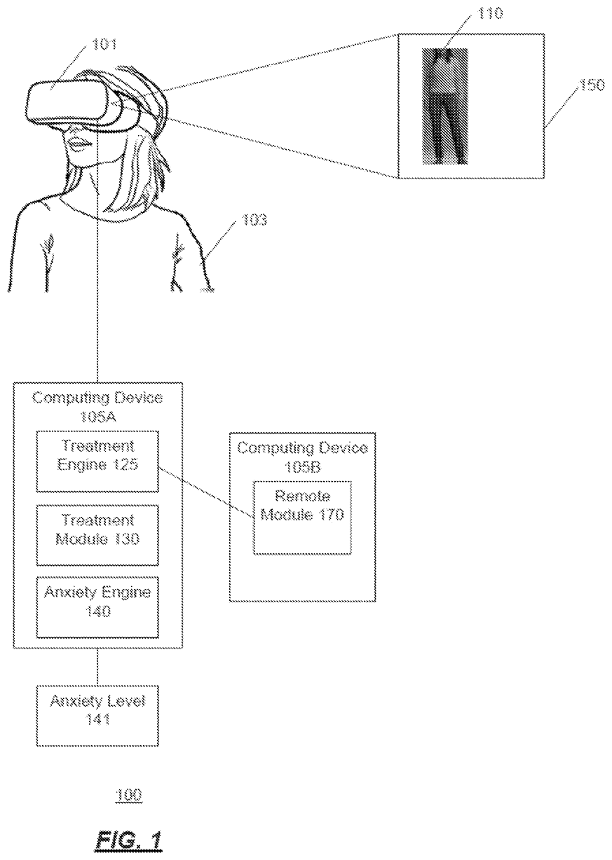

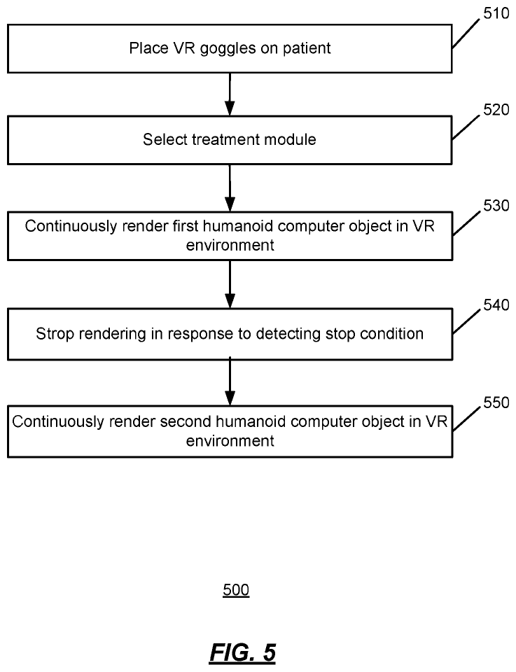

FIG. 1 is an example system for treating one or more fears related to an eating disorder. As shown, the system 100 may include one or more of goggles 101 and one or more computing devices 105 (e.g., the computing devices 105A and 105B). Each of the goggles 101 and the computing devices 105 may be partially implemented by one or more general purpose computing devices such as the computing device 500 illustrated in FIG. 5.

The goggles 101 (also referred to as glasses or headsets) may be virtual reality (VR) goggles or augmented reality (AR) goggles. Where the goggles 101 are VR goggles, the goggles 101 may render a complete virtual environment 150 that is displayed to a wearer of the goggles 101. The wearer may then interact with one or more virtual objects in the virtual environment 150 using one or more controls associated with the goggles 101 (not shown). Where the goggles 101 are AR goggles, the wearer may see the real world through the goggles 101, but the goggles 101 may render one or more virtual objects into the field of view of the wearer such that the virtual objects appear to exist in the real world. While VR and AR are different, for purposes of simplification, any features or embodiments described herein with respect to VR may be assumed to apply equally to AR, and vice versa.

The system 100 may be used to provide one or more treatments to a patient 103. During treatment, the patient 103 may wear the goggles 101 which may be connected to the computing device 105A. The computing device 105A may be implemented using a variety of computing devices including, but not limited to, desktop, laptop, and personal computing devices, tablet computing devices, smart phones, and videogame consoles, for example. Depending on the embodiment, the goggles 101 may connect to the computing device 105A using a wired connection (e.g., USB, HDMI, fiberoptic, and ethernet) or a wireless connection (e.g., WiFi or Bluetooth).

The computing device 105A may include a treatment engine 125, a treatment module 130, and an anxiety engine 140. At a high level, the treatment engine 125 may control the goggles 101 being worn by the patient 103 and may facilitate the execution of the treatment module 130 that is selected by a treatment provider (e.g., doctor, therapist, or other medical or mental health provider).

The treatment module 130 may include a treatment for a particular phobia or fear associated with an eating disorder. Example fears may include a fear of gaining weight, a fear of uncomfortable bodily sensations (e.g., gastrointestinal sensations, bloating, fullness, jiggly body, rubbing body areas, etc.), a fear of being judged, a fear of eating in front of others, and the fear of being perceived as fat. Other fears may be supported. In some embodiments, a treatment module 130 may be stored, downloaded, or loaded into the computing device 105A for each fear that the system 100 treats. Accordingly, the system 100 can be continuously updated as new fears are identified, or new treatment approaches are identified.

The anxiety engine 140 may monitor the anxiety level 141 of the patient 103 being treated. The anxiety engine 140 may determine the anxiety level 141 of the patient 103 using a questionnaire, where the patient 103 is periodically asked to rate or score their current anxiety level 141. An example of such a system is the Subjective Units of Distress Scale (SUDS) where a patient 103 is asked to assign a number of 0-100 to their perceived anxiety level.

Alternatively, the anxiety engine 140 determine the anxiety level 141 using one or more physiological signals of the patient 103. For example, the anxiety engine 140 may use information collected about the patient 103 by the goggles 101 (or other device or sensor) such as heart rate, eye gaze, and respiration rate, and may use the information to determine an anxiety level 141 of the patient 103.

The computing device 105B may be used by the treatment provider to control the progression of the treatment being received by the patient 103. The computing device 105B may include a remote module 170 that interfaces with the treatment engine 125 and allows the treatment provider to select the particular treatment module 130 and to provide instructions to the treatment engine 125 that control the progress of the treatment with respect to the patient 103. A suitable computing device 105B includes desktop, laptop, and personal computing devices, tablet computing devices, smart phones, and videogame consoles, for example.

In some embodiments, the treatment provider and the patient 103 may be located in the same room. In these embodiments, the computing devices 105A and 105B may communicate directly using one or more wireless or wired communication technologies. Alternatively, the computing devices 105A and 105B may communicate via a LAN. In addition, the computing devices 105A and 105B may be the same computing device.

In other embodiments, the treatment provider and the patient 103 may be at different locations with the computing device 105A and the goggles 101 with the patient 103 at a first location, and the computing device 105B with the treatment provider at a second location. In these embodiments, the computing devices 105A and 105B may communicate via the internet or other network.

To facilitate the treatment of one or more fears related to an eating disorder, the treatment engine 125 may be used to create what is referred to herein as a humanoid computer object 110. A humanoid computer object 110 as used herein is a digital representation or rendering of the patient 103. The humanoid computer object 110 may be placed into the virtual environment 150 displayed by the goggles 101, and the patient 103 may be able to view the humanoid computer object 110 from a variety of positions and angles. For example, the patient 103 can use controls associated with the goggles 101 to turn the humanoid computer object 110 so that the patient 103 can view the humanoid computer object 110 from different angles, perspectives, and distances, or the patient 103 may be able to walk around the object 110 in the virtual environment 150.

The treatment engine 125 may provide one or more software tools that may be used to create the humanoid computer object 110 for a patient 103. Depending on the embodiment, the treatment provider may submit a picture of the patient 103 along with measurements and characteristics of the patient 103 such as height, weight, age, race, etc., and the treatment engine 125 may generate a humanoid computer object 110 based on the picture and measurements. Alternatively, the treatment provider may submit the patient measurements and characteristics and the treatment engine 125 may generate the humanoid computer object 110 without a photograph. Another alternative is that the patient or treatment provider may personalize the humanoid computer object 110 to the patient by sculpting the different body areas and by personalized selection in a graphical user interface. In some embodiments, the patient may be 3D scanned or otherwise measured, and the measurements may be used to generate the humanoid computer object 110.

The treatment engine 125 may further allow the treatment provider to customize the humanoid computer object 110 by selecting hair styles, clothing, and accessories such as glasses, shoes, and handbags, for example. The treatment provider may customize the humanoid computer object 110 such that it is recognizable to the patient 103 as a representation of themselves. Any method for generating a humanoid computer object 110 such as an avatar may be used.

After generating the initial humanoid computer object 110, the treatment engine 125 may further generate plurality of other humanoid computer objects 110, each with a different weight value. Each humanoid computer object 110 may have a weight value that is greater than (or less than) a previous humanoid computer object 110 by a weight offset. The offset may be percentage (e.g., 5%, 10%, or 15%), or may be a number of pounds (e.g., 5 pounds, 10 pounds, or 15 pounds). The goal of each humanoid computer object 110 may be for the patient 103 to see themselves at different weights and to make the patient 103 comfortable with seeing themselves with different weights. As will be described further below, the various humanoid computer objects 110 may be displayed to the patient 103 during treatment to help reduce the patient's 103 fear of gaining weight.

In some embodiments, the treatment engine 125 may allow the patient (or treatment provider) to personalize the patient experience by modifying the humanoid computer object 110 to change its weight, color, size, shape, color, and/or gender to target specific eating disorder related fears. In addition, the patient (or treatment provider) may target certain areas (e.g., belly, thighs, cheeks, and chin) of the humanoid computer object 110 to increase weight or to add or modify an amount of jiggle associated with the targeted area. Further, the treatment engine 125 may allow the patient (or treatment provider) to add gamification features such as drinking coinciding with fullness, bloating, or other bodily sensations.

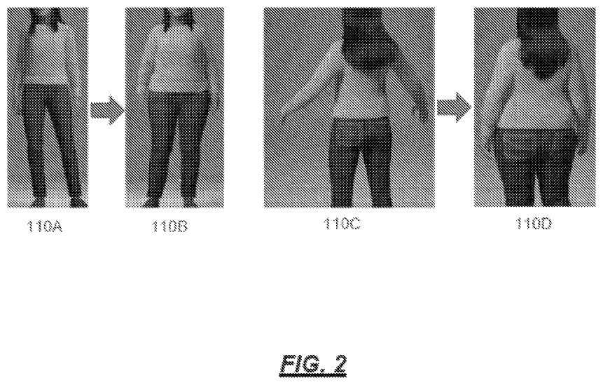

FIG. 2 is an illustration of several example humanoid computer objects 110 (e.g., the objects 110A, 110B, 110C, and 110D). The humanoid computer objects may represent the same patient 110 with different weight values. For example, the humanoid computer objects 110A and 110C may be different views of the same patient 103 with weight values that are close to the actual weight of the patient 103. The humanoid computer objects 110B and 110D may be different views of the same patient 103 with weight values are greater than the actual weight of the patient 103 by a weight offset.

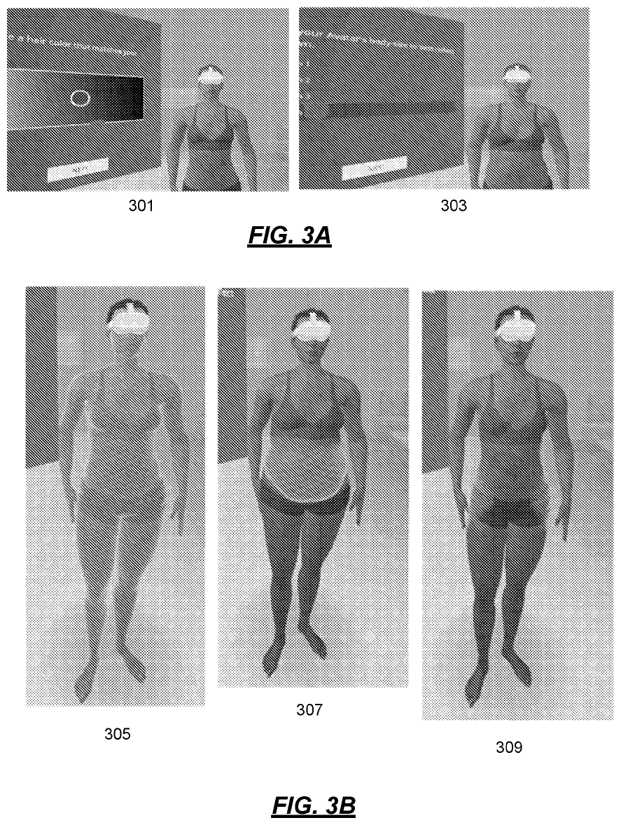

FIGS. 3A, 3B, and 3C are illustrations of example images of a virtual environment that may be used to render the humanoid computer objects. With reference to FIG. 3A is shown two screenshots 301 and 303 from a virtual environment where a patient 103 is creating their humanoid computing object. The patient 103 may select various attributes of their humanoid computer object. As shown in the screenshot 301, the patient 103 is using a slider, point and click, or hand gestures to select a hair color for their humanoid computer object. As shown in the screenshot 303, the patient is using a slider, point and click, or hand gestures to adjust the body size of their humanoid computer object.

With reference to FIG. 3B, the patient may target particular areas of the body for adjustment. As shown in the screenshot 305, the patient 103 a grid is shown over the humanoid computing object. The patient may adjust the grid to specify the part of the body that they would like to change (e.g., increase or decrease size, or add jiggle). As shown in the screenshot 307, the patient 103 has adjusted the grid to select their belly. As shown in the screenshot 309, the patient 103 has adjusted the grid to select their thighs. Depending on the embodiment, the selected area of the body may be targeted for weight gain according to the treatment module 130 selected by the patient 103 or treatment provider.

Returning to FIG. 1, the treatment provider may begin a treatment session for the patient 103 by selecting a corresponding treatment module 130 using the computing device 105B and may instruct the patient 103 to begin wearing the goggles 101.

Before or after the patient 103 is wearing the goggles 101, the treatment engine 125 may use the anxiety engine 140 to determine a baseline anxiety level 141 for the patient 103. This baseline anxiety level 141 may be used by the treatment engine 125 to determine if a later measured anxiety level 141 is high or low for the patient 103.

The anxiety engine 140 may determine the baseline anxiety level 141 by asking the patient 103 one or more questions directed to determining an anxiety level 141. Alternatively, the treatment provider may ask the patient 103 the one or more questions and may provide the answers to the anxiety engine 140 through the computing device 105B. Alternatively, or additionally, the anxiety engine 140 may determine the anxiety level 141 of the patient 103 using one or more physiological signals (e.g., pulse, or respiration rate) received from the patient 103.

Once the baseline anxiety level 141 of the patient is determined, the treatment engine 125 may cause one or more humanoid computer objects 110 to be displayed to the patient 103 in the virtual environment 150. Initially, the displayed humanoid computer objects 110 may have weight values that are based on the current weight of the patient 103 and may have a similar appearance as the patient 103. Where multiple humanoid computer objects 110 are displayed, each humanoid computer object 110 may be the same, or some of the humanoid computer objects 110 may have different hairstyles and outfits. Each hairstyle or outfit may correspond to a hairstyle or outfit used by the patient 103 or selected by the patient 103.

The patient 103 may interact with the humanoid computer objects 110 in the virtual environment 150. Depending on the embodiment, the humanoid computer objects 110 may be rendered or displayed so that the eyes of the humanoid computer objects 110 are not visible to the user. In one embodiment, an angle of a camera that represents the field of view of the goggles 101 in the virtual environment 150 may be limited such that the patient 103 is unable to see the eyes of the humanoid computer objects 110. For example, as can be seen in FIG. 2, none of eyes of the humanoid computer objects are visible due to the angle of the camera. Alternatively, or additionally, the faces (i.e., facial regions) of the humanoid computer objects 110 may be obscured with a mosaic or other object, while being able to see areas of the face (e.g., cheeks, neck, etc.) that may be problematic to the patient (e.g., chubby cheeks, double neck). During the treatment the patient 103 may be able to view the humanoid computer objects 110 from a variety of angles including directly above and looking down.

At some point after displaying the humanoid computer objects 110 to the patient 103, the treatment engine 125 may use the anxiety engine 140 to determine a current anxiety level 141 of the patient 103. When the current anxiety level 141 is within a threshold percentage of the baseline anxiety level 141, the treatment engine 125 may determine that the patient 103 is not experiencing high anxiety with respect to the humanoid computer objects 110 that are being displayed.

Once the current anxiety level 141 of the patient 103 is determined to be safe, the treatment engine 125 may proceed to a next phase of the treatment where different humanoid computer objects 110 are displayed and rendered in the virtual environment 150. Each displayed different humanoid computer object 110 may have a weight value that is higher than the weight values of the previously displayed humanoid computer objects 110. Depending on the embodiment or treatment module selected, the different humanoid computer objects 110 may have weight gain in certain target areas or body parts, may have increased movement or jiggle in certain areas or body parts, or may show certain signs of uncomfortable feelings such as bloating, for example.

The patient 103 may view and interact with the humanoid computer objects 110 having the increased weight values, or other changes, in the virtual environment 150, which may allow the patient 103 to see how they might look were they to gain a similar amount of weight, or have the same changes, as the humanoid computer objects 110. In some embodiments, each of the different humanoid objects 110 has a weight value that has been increased by a same weigh offset. Alternatively, some of the different humanoid objects 110 may have weight values that were increased by different offsets.

While the patient 103 interacts with the humanoid computer objects 110 in the environment 150, the treatment provider may ask the patient 103 questions and may encourage the patient 103 to interact with the humanoid computer objects 110. In addition, the treatment engine 125 may continue to monitor the anxiety level 141 of the patient 103 and to compare it with the baseline anxiety level 141 of the patient 103.

Where gamification is used, the patient may be encouraged to take actions (either real or virtually) such as drinking or eating. In response to the drinking or eating, the humanoid objects 110 may be modified to show results of the eating or drinking such as bloating or weight gain. The anxiety of the patient may be continued to be monitored.

Because the patient 103 has eating disorder specific fears it is expected that the anxiety level 141 of the patient 103 will initially increase past the threshold percentage of the baseline anxiety level 141. However, if the anxiety level 141 of the patient 103 gets too high or remains outside of the threshold percentage for too long, the treatment engine 125 may end the treatment.

In some embodiments, once the current anxiety level 141 of the patient 103 falls below the threshold percentage of the baseline anxiety level 141, the treatment engine 125 may again replace the currently displayed humanoid computer objects 110 with new humanoid computer objects 110 that have even greater weight offsets, or other changes. This process may continue until a time allotted to the treatment has expired or as determined by the treatment provider. During the process other changes may be made to the humanoid computer object 110 (e.g., increase jiggles or other changes), depending on the particular phobia or fear that is being addressed.

In some embodiments, rather than just displaying humanoid computer objects 110 to the user with weight values that are greater than the current weight of the patient 103, the treatment engine 125 may generate humanoid computer objects 110 with weight values that are both higher and lower than the current weigh of the patient 103. The humanoid computer objects 110 including the humanoid computer objects 110 with the lower and higher weight values may then be presented to the patient 103 in groups in the environment 150 while the anxiety level 141 of the patient 103 is monitored. The treatment may continue until the measured anxiety level 141 shows that the patient 103 has become comfortable with viewing themselves with a variety of different weights.

Similarly, rather than just displaying the displaying humanoid computer objects 110 to the user with modified weight values, the treatment engine 125 may display humanoid computer objects 110 with varying levels of jiggly or enlarged independent areas. These areas may be selected by the treatment provider and the anxiety of the patient may be monitored as the patient is presented with different levels of modification. The treatment may continue until the measured anxiety level 141 shows that the patient 103 has become comfortable with viewing themselves with different sized areas or levels of jiggle.

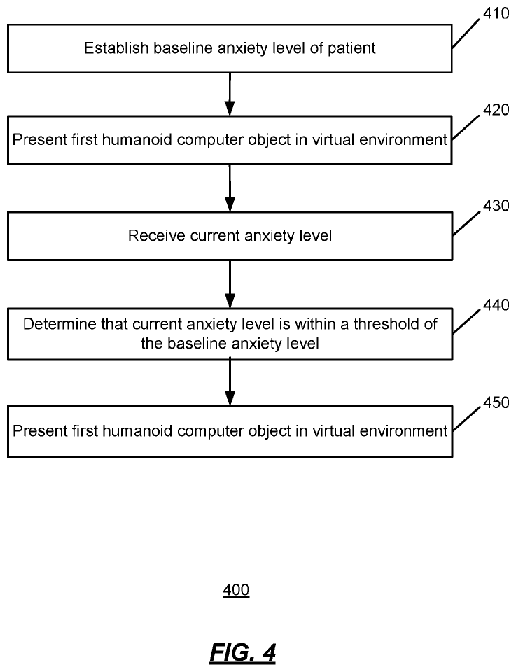

FIG. 4 is an illustration of FIG. 3 is an illustration of a method 400 for treating one or more fears associated with an eating disorder. The method 400 may be performed by the system 100.

At 410, a baseline anxiety level of a patient is determined. The baseline anxiety level 141 of patient 103 may be determined by an anxiety engine 140 of the system 100. The patient 103 may be beginning or may be about to begin treatment for one or more fears related to an eating disorder. In this example, the fear is the fear of gaining weight. Other fears may be supported.

In some embodiments, the baseline anxiety level 141 of the patient 103 may be determined by the anxiety engine 140 using a variety of techniques such as having the treatment provider ask the patient 103 to assign a score to their anxiety or to ask the patient a series of questions related to their anxiety. The anxiety engine 140 may then determine the anxiety level 141 of the patient 103 based on the answers to the questions. Alternatively, or additionally, the anxiety engine 140 may measure one or more physiological signals of the patient 103 such as heart rate to determine the baseline anxiety level 141. Other methods for measuring anxiety may be used.

At 420, a first humanoid computer object is presented in a virtual environment. The first humanoid computer object 110 may be presented in a virtual environment 150 and rendered to the patient 103 by the goggles 101. The first virtual humanoid object 110 may have an associated wight value and a general likeness that is similar to the current weight and likeness of the patient 103. Depending on the embodiment, the first humanoid computing object 110 may have been generated by the treatment engine 125 using a photograph of the patient 103 and information about the patient such as weight, height, sex, race etc. Additionally, or alternatively, the treatment engine 125 may generate the first humanoid computing object 110 solely based on parameters provided by the treatment provider or the patient 103, or a 3D scan of the patient.

In some embodiments, the first humanoid computing object 110 may be rendered and presented so that the face (or eyes) of the first humanoid computer object 110 is obscured. For example, a viewing perspective or angle of a camera in the virtual environment 150 may be restricted such that the patient 103 cannot see the head or face of the first humanoid computer object 110. Alternatively, or additionally, some or all of the face or head of the first humanoid computer object 110 may be obscured using an object (e.g., goggles), mosaic, pixelization, or a blurring or other distorting effect. In some embodiments, only the eyes of the humanoid object 110 may be hidden so the patient can see ‘problematic areas’ such as chubby cheeks, double or triple chins, and neck fat without fixating on the eyes.

At 430, a current anxiety level of the patient is determined. The current anxiety level of the patient 103 may be determined by the anxiety engine 140. The current anxiety level 141 may be determined after the patient 103 has been observing the first humanoid computer object 110 in the environment 150 for some predetermined amount of time. Alternatively, or additionally, the anxiety level 141 of the patient 103 may be continuously determined by the anxiety engine 140 while the patient 103 views the first humanoid computer object 110.

At 440, that the current anxiety level is within a threshold of the baseline anxiety level is determined. The determination may be made by the treatment engine 125 by comparing the current anxiety level 141 and the baseline anxiety level 141 to a threshold. The threshold may be set by the treatment provider. Example thresholds may be 5%, 10%, 15%, etc. That the current anxiety level 141 is within the threshold of the baseline anxiety level 141 may indicate that the patient 103 is calm and is not bothered by viewing the first humanoid computer object 110.

At 450, a second humanoid computer object is presented. The second humanoid computer object 110 may be presented in the virtual environment 150 and rendered to the patient 103 by the goggles 101. The second humanoid computer object 110 may replace the first humanoid computer object 110 in the environment 150 and may have been presented in response to the current anxiety level 141 being within the threshold.

The second humanoid computer object 110 may be substantially similar to the first humanoid computer object 110 but may be rendered have a larger size or weight value than the first humanoid computer object 110. While the first humanoid computer object 110 was rendered to appear similar in weight and size as the patient 103, the second humanoid computer object 110 is rendered to appear as if the patient 103 has gained a selected amount of weight. Depending on the embodiment, the amount of weight increase for the second humanoid computer object 110 may be a set amount of weight (e.g., 10 pounds), or may be a percentage of their current weight (e.g., ten percent). In another embodiment, the second humanoid computer object 110 may be substantially similar to the first humanoid computer object 110 but may be rendered with a size of a specific area increase (e.g., larger thighs, or cheeks). In addition, certain parts or areas of the second humanoid computer object 110 may have an increased jiggle or movement when compared to the first object 110.

Depending on the embodiment, the system 100 may monitor the current anxiety level 141 of the patient while viewing the second humanoid computer object 110. The patient 110 may continue to view the second humanoid computer object 110 until the treatment session has ended and/or the current anxiety level 141 is within the threshold of the baseline anxiety level 141. In some embodiments, when the current anxiety level 141 is within the threshold of the baseline anxiety level 141, a third humanoid computer object 110 with a weight that is larger than the second humanoid computer object 110 (or other changes) may be rendered and displayed.

FIG. 5 is an illustration of a method 500 for treating one or more fears associated with an eating disorder. The method 500 may be performed by the system 100.

At 510, goggles are placed on a patient. The goggles 101 (VR or AR goggles) may be and may be part of a treatment system 100 and may be placed on the head of the patient 103 by a treatment provider or the patient 103 themselves. The goggles 101 may be connected to a computing device 105A that is part of the treatment system 100. The goggles 101 may render a VR environment 150 (or AR environment 150) to the patient 103.

The patient 103 may be under the care of the treatment provider and may have put on the goggles 101 to receive a treatment from the treatment provider. The treatment may be for a fear associated with an eating disorder. In some embodiments, the patient 103 and the treatment provider are in the same room. Alternatively, the patient 103 and the treatment provider are at different locations and the computing devices 105A and 105B may be connected via a network such as the internet.

At 520, a treatment module is selected. The treatment module 130 may be selected by the treatment provider using a computing device 105B associated with the treatment system 100. The selected treatment module 130 may be for the treatment of a fear of gaining weight. Other fears may be supported.

At 530, a first humanoid computer object is continuously rendered in the virtual environment 150. The first humanoid computer object 110 may be rendered by the treatment engine 125 and/or the goggles 101. The first humanoid object may be rendered to appear similar to the patient 103 with a weight value that is based on the actual weight of the patient 103. Other changes may be supported such as increased jiggle, bloating, or other qualities. The patient 103 may be able to view and interact with the first humanoid object 110 in the virtual environment 150. In some embodiments, the first humanoid computer object 110 may be rendered with an obscured face or part of the face (e.g., eyes), or the patient 103 may be restricted to view the virtual environment from camera positions or angles that prevent the patient 103 from viewing the face, or part of the face (e.g., eyes) of the first humanoid computer object.

At 540, a stop condition is detected. The stop condition may be detected by the treatment engine 125. The stop condition may be a variety of conditions including an anxiety level 141 of the patient 103 being within a baseline anxiety level 141 indicating that the patient 103 is not anxious or overly anxious, and an amount of time being exceeded. Other stop conditions may be supported.

At 550, a second humanoid computer object is continuously rendered in the virtual environment 150. The second humanoid object may be rendered by the treatment engine 125 and/or the goggles 101. Like the first humanoid object, the second humanoid computer object may be rendered to appear similar to the patient 103. However, the second humanoid computer object may have a weight value that is based on the actual weight of the patient 103 plus some weight offset. The weight offset may be a percentage of the actual weight of the patient 103 (e.g., 5%) or may be a fixed number of points (e.g., 5 pounds). The weight offset may be selected by the treatment provider. Other changes to the humanoid computer object may be made depending on the fear being treated.

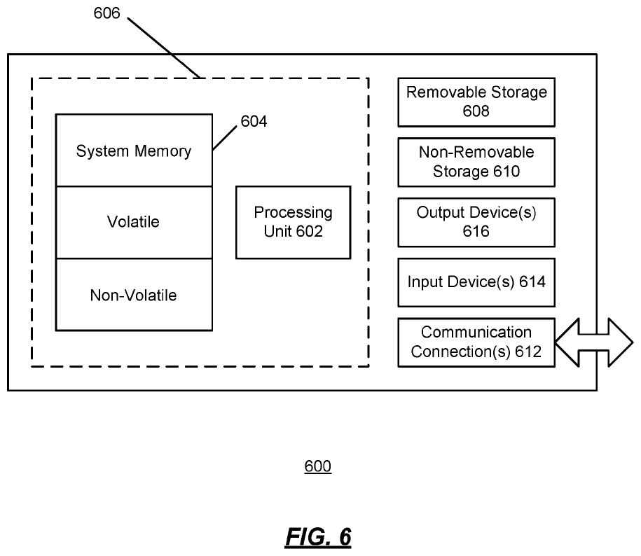

FIG. 6 shows an example computing environment in which example embodiments and aspects may be implemented. The computing device environment is only one example of a suitable computing environment and is not intended to suggest any limitation as to the scope of use or functionality.

Numerous other general purpose or special purpose computing devices environments or configurations may be used. Examples of well-known computing devices, environments, and/or configurations that may be suitable for use include, but are not limited to, personal computers, server computers, handheld or laptop devices, multiprocessor systems, microprocessor-based systems, network personal computers (PCs), minicomputers, mainframe computers, embedded systems, distributed computing environments that include any of the above systems or devices, and the like.

Computer-executable instructions, such as program modules, being executed by a computer may be used. Generally, program modules include routines, programs, objects, components, data structures, etc. that perform particular tasks or implement particular abstract data types. Distributed computing environments may be used where tasks are performed by remote processing devices that are linked through a communications network or other data transmission medium. In a distributed computing environment, program modules and other data may be located in both local and remote computer storage media including memory storage devices.

With reference to FIG. 6, an example system for implementing aspects described herein includes a computing device, such as computing device 600. In its most basic configuration, computing device 600 typically includes at least one processing unit 602 and memory 604. Depending on the exact configuration and type of computing device, memory 604 may be volatile (such as random access memory (RAM)), non-volatile (such as read-only memory (ROM), flash memory, etc.), or some combination of the two. This most basic configuration is illustrated in FIG. 6 by dashed line 606.

Computing device 600 may have additional features/functionality. For example, computing device 600 may include additional storage (removable and/or non-removable) including, but not limited to, magnetic or optical disks or tape. Such additional storage is illustrated in FIG. 6 by removable storage 608 and non-removable storage 610.

Computing device 600 typically includes a variety of computer readable media. Computer readable media can be any available media that can be accessed by the device 600 and includes both volatile and non-volatile media, removable and non-removable media.

Computer storage media include volatile and non-volatile, and removable and non-removable media implemented in any method or technology for storage of information such as computer readable instructions, data structures, program modules or other data. Memory 604, removable storage 608, and non-removable storage 610 are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device 600. Any such computer storage media may be part of computing device 600.

Computing device 600 may contain communication connection(s) 612 that allow the device to communicate with other devices. Computing device 600 may also have input device(s) 614 such as a keyboard, mouse, pen, voice input device, touch input device, etc. Output device(s) 616 such as a display, speakers, printer, etc. may also be included. All these devices are well known in the art and need not be discussed at length here.

It should be understood that the various techniques described herein may be implemented in connection with hardware components or software components or, where appropriate, with a combination of both. Illustrative types of hardware components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc. The methods and apparatus of the presently disclosed subject matter, or certain aspects or portions thereof, may take the form of program code (i.e., instructions) embodied in tangible media, such as floppy diskettes, CD-ROMs, hard drives, or any other machine-readable storage medium where, when the program code is loaded into and executed by a machine, such as a computer, the machine becomes an apparatus for practicing the presently disclosed subject matter.

Although example implementations may refer to utilizing aspects of the presently disclosed subject matter in the context of one or more stand-alone computer systems, the subject matter is not so limited, but rather may be implemented in connection with any computing environment, such as a network or distributed computing environment. Still further, aspects of the presently disclosed subject matter may be implemented in or across a plurality of processing chips or devices, and storage may similarly be effected across a plurality of devices. Such devices might include personal computers, network servers, and handheld devices, for example.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. An augmented-reality or virtual-reality system comprising
a processor;
a memory operatively coupled to the processor, the memory having instructions stored thereon for treating eating disorders, wherein execution of the instructions by the processor, cause the processor to:
continuously render a first humanoid computer object in an augmented- or virtual-reality environment selected from a plurality of humanoid bodies defined at least by a gender type, body type, skin pigment, and associated weight, including a first selected body having an associated first weight value and a likeliness in appearance to the user;
stop rendering of the first humanoid computer object based on a stop condition associated with an anxiety level value or score received from the user; and
continuously render a second humanoid computer object in the augmented- or virtual-reality environment selected from the plurality of humanoid bodies, including a second selected body having an associated second weight value and a likeliness in appearance to the user, wherein the second weight value is greater than the first weight value.

2. The augmented-reality or virtual-reality system of claim 1, wherein the user is sequentially presented a series of humanoid computer objects in the augmented- or virtual-reality environment, the series of humanoid computer objects having (i) a predefined number of selected bodies having a low weight value and a likeliness in appearance to the user, (ii) a current predefined number of selected bodies having a current weight value and a likeliness in appearance to the user, and (iii) a predefined number of selected bodies having a high weight value and a likeliness in appearance to the user.

3. The augmented-reality or virtual-reality system of claim 1, wherein the low weight value, the current weight value, and the high weight value are selectable based on a percentage of the current weight of the patient.

4. The augmented-reality or virtual-reality system of claim 1, wherein the low weight value, the current weight value, and the high weight value are selectable based on a pre-defined offset of the current weight of the patient.

5. The augmented-reality or virtual-reality system of claim 1, wherein the first humanoid computer object is presented from a view of a camera defined in the augmented- or virtual-reality environment, wherein the camera is co-located to the first humanoid computer object.

6. The augmented-reality or virtual-reality system of claim 5, wherein the camera has a pre-defined height level that limits presentation of the first humanoid computer object to exclude a facial region of the first humanoid computer object.

7. The augmented-reality or virtual-reality system of claim 1, wherein the first humanoid computer object has a blocking mosaic over a facial region of the first humanoid computer object.

8. The augmented-reality or virtual-reality system of claim 1, wherein the system comprises an AR-VR goggle.

9. A method of conducting an eating disorder therapy, the method comprising:
establishing a baseline anxiety level value or score for a patient at a commencement of an eating disorder therapy;
presenting, via a processor executing an augmented- or virtual-reality environment, to a patient, a first humanoid computer object in the augmented- or virtual-reality environment selected from a plurality of humanoid bodies defined at least by a gender type, body type, skin pigment, and associated weight, wherein the first humanoid computer object includes a first selected body having a likeliness in appearance to the patient;
receiving, via the processor, a selection of a treatment module of a plurality of treatment modules, wherein each treatment module treats a different fear associated with an eating disorder;
receiving an anxiety level value or score from the patient and upon the anxiety level value or score being within a pre-defined threshold of the baseline anxiety level value or score, stop presentation of the first humanoid computer object; and
presenting, via the processor, a second humanoid computer object in the augmented- or virtual-reality environment selected, wherein the second humanoid computer object includes at least one change based on the fear associated with the selected treatment module.

10. The method of claim 9, wherein the fear associated with the selected treatment module is a fear of weight gain, and at the at least one change to the second humanoid computer object comprises an increased weight of the second humanoid computer object in contrast with the first humanoid computer object.

11. The method of claim 9, wherein the fear associated with the selected treatment module is a fear of bodily sensations, and at the at least one change to the second humanoid computer object comprises one or more of increased bloating or of increased jiggling in contrast with the first humanoid computer object.

12. The method of claim 9, wherein the fear associated with the selected treatment module is a fear of weight gain, and the at least one change to the second humanoid computer object comprises a weight gain to selected body part of area of the second humanoid computer object in contrast with the first humanoid computer object.

13. The method of claim 9, further comprising:
detecting, via the processor, that that the user has consumed a product; and
in response to the detecting, presenting, via the processor, a third humanoid computer object in the augmented- or virtual-reality environment selected, wherein the third humanoid computer object includes at least one change based on the fear associated with the selected treatment module.

14. A non-transitory computer readable medium having instructions stored thereon for a virtual-reality software for eating disorder therapy, wherein execution of the instructions by the processor causes the processor to:
continuously render a first humanoid computer object in an augmented- or virtual-reality environment selected from a plurality of humanoid bodies defined at least by a gender type, body type, skin pigment, and associated weight, including a first selected body having an associated first weight value and a likeliness in appearance to the user;
stop rendering of the first humanoid computer object based on a stop condition associated with an anxiety level value or score received from the user; and
continuously render a second humanoid computer object in the augmented- or virtual-reality environment selected from the plurality of humanoid bodies, including a second selected body having an associated second weight value and a likeliness in appearance to the user, wherein the second weight value is greater than the first weight value.

15. The computer readable medium of claim 14, wherein the user is sequentially presented a series of humanoid computer objects in the augmented- or virtual-reality environment, the series of humanoid computer objects having (i) a predefined number of selected bodies having a low weight value and a likeliness in appearance to the user, (ii) a current predefined number of selected bodies having a current weight value and a likeliness in appearance to the user, and (iii) a predefined number of selected bodies having a high weight value and a likeliness in appearance to the user.

16. The computer readable medium of claim 14, wherein the low weight value, the current weight value, and the high weight value are selectable based on a percentage of the current weight of the patient.

17. The computer readable medium of claim 14, wherein the low weight value, the current weight value, and the high weight value are selectable based on a pre-defined offset of the current weight of the patient.

18. The computer readable medium of claim 14, wherein the first humanoid computer object is presented from a view of a camera defined in the augmented- or virtual-reality environment, wherein the camera is co-located to the first humanoid computer object.

19. The computer readable medium of claim 18, wherein the camera has a pre-defined height level that limits presentation of the first humanoid computer object to exclude a facial region of the first humanoid computer object.

20. The computer readable medium of claim 14, wherein the first humanoid computer object has a blocking mosaic over a facial region of the first humanoid computer object.
